(19) United States
(12) Patent Application Publication
(10) Pub. No.: US 2021/0169422 A1
(43) Pub. Date: Jun. 10, 2021

(54) ROBOT FOR MEDICAL ASSISTANCE

(71) Applicants: Kamran Mashaw, Beaverton, OR (US); Arianna Mashaw, Beaverton, OR (US)

(72) Inventors: Kamran Mashaw, Beaverton, OR (US); Arianna Mashaw, Beaverton, OR (US)

(21) Appl. No.: 17/177,701

(22) Filed: Feb. 17, 2021

(51) Int. Cl. A61B 5/00; A61B 5/0205; A61L 2/26; A61L 2/10; A61L 2/18; B25J 11/00; H04R 1/02; H04R 1/08

(52) U.S. Cl. CPC A61B 5/6887; A61B 5/02055; A61B 5/7445; A61B 5/002; A61L 2/26; A61B 5/02233; A61L 2/18; B25J 11/009; H04R 1/028; H04R 1/08; A61L 2/10

(57) ABSTRACT

A robot that can be remotely controlled for taking the vitals of a patient without the direct assistance of the clinical staff. The robot having a head, a body, and wheels. To the body of the robot can be coupled diagnostic devices positioned to be within the reach of a patient. The robot includes a camera, speakers, microphone, and a display. The robot can also include a central processing unit for controlling the different components of the robot and remotely connecting to other computing devices. The diagnostic devices can include the blood pressure monitoring device having a cuff coupled to the body, such as a patient can insert his hand in the cuff. The diagnostic devices can also include a pulse oximeter and an IR thermometer.

### FIELD OF INVENTION

The present invention relates to a robot for medical assistance, and more particularly, the present invention relates to a robot for taking the vitals of a patient.

### BACKGROUND

Hospital-acquired infections, also known as nosocomial infections, are infections contracted under hospital settings. Doctors and nurses who treat the patients are at a greater risk of contracting the infections. Particularly, in the intensive care units where highly infectious diseases are treated, are the source for the maximum number of hospital-acquired infections. Nurses must frequently take the vitals of the patients, which exposes them to a greater risk of contracting the infection. The nurses in turn may become a source of the spread of the infection to other patients and people. Doctors and nurses generally use face masks, gloves, or personal protective kits for protection, however, such protective kits offer limited protection. Certain infectious diseases, such as SARS or COVID-19 are highly infectious and despite all the precautions, may spread to other persons.

Thus, a need is appreciated for a medical aid that can assist medical professionals in taking vitals of a patient and other functions of a nurse.

### SUMMARY OF THE INVENTION

The principal object of the present invention is therefore directed to a robot for medical assistance.

It is another object of the present invention that the robot can take vital of a patient independently.

It is still another object of the present invention that the robot can sanitize its body.

It is yet another object of the present invention that the robot can be remotely controlled.

It a further object of the present invention that the robot allows videoconferencing with medical staff.

It is an additional object of the present invention that the robot is economical to manufacture.

In one aspect, disclosed is a robot that can be remotely controlled for taking vitals of a patient without the need for the clinical staff to come in contact with the patient. The robot can have a face resembling the face of clinical staff, and a body and wheels. To the body of the robot can be coupled diagnostic devices positioned to be within the reach of a patient. A camera can be coupled to the face, maybe two cameras can be coupled forming the eyes of the robot. A speaker can be coupled below the eyes forming a mouth. The robot can include a central processing unit for controlling the different components of the robot and remotely connecting to other computing devices. The camera and the speakers can provide video conferring between the patient and the clinical staff. The diagnostic devices can include the blood pressure monitoring device having a cuff coupled to an outer surface of the body, such as a patient can insert his arm in the cuff. The diagnostic devices can also include a pulse oximeter and an IR thermometer.

### BRIEF DESCRIPTION OF DRAWING

The accompanying figures, which are incorporated herein, form part of the specification and illustrate embodiments of the present invention. Together with the description, the figures further explain the principles of the present invention and to enable a person skilled in the relevant arts to make and use the invention.

FIG. 1 shows a perspective view of the robot, according to an exemplary embodiment of the present invention.

FIG. 2 is an environmental diagram showing the robot connected to a clinical staff device and another device through a network, according to an exemplary embodiment of the present invention.

### DETAILED DESCRIPTION

Subject matter will now be described more fully hereinafter with reference to the accompanying drawings, which form a part hereof, and which show, by way of illustration, specific exemplary embodiments. Subject matter may, however, be embodied in a variety of different forms and, therefore, covered or claimed subject matter is intended to be construed as not being limited to any exemplary embodiments set forth herein; exemplary embodiments are provided merely to be illustrative. Likewise, a reasonably broad scope for claimed or covered subject matter is intended. Among other things, for example, the subject matter may be embodied as methods, devices, components, or systems. The following detailed description is, therefore, not intended to be taken in a limiting sense.

The word “exemplary” is used herein to mean “serving as an example, instance, or illustration.” Any embodiment described herein as “exemplary” is not necessarily to be construed as preferred or advantageous over other embodiments. Likewise, the term “embodiments of the present invention” does not require that all embodiments of the invention include the discussed feature, advantage, or mode of operation.

The terminology used herein is for the purpose of describing particular embodiments only and is not intended to be limiting of embodiments of the invention. As used herein, the singular forms “a”, “an” and “the” are intended to include the plural forms as well, unless the context clearly indicates otherwise. It will be further understood that the terms “comprises”, “comprising,”, “includes” and/or “including”, when used herein, specify the presence of stated features, integers, steps, operations, elements, and/or components, but do not preclude the presence or addition of one or more other features, integers, steps, operations, elements, components, and/or groups thereof.

The following detailed description includes the best currently contemplated mode or modes of carrying out exemplary embodiments of the invention. The description is not to be taken in a limiting sense but is made merely for the purpose of illustrating the general principles of the invention, since the scope of the invention will be best defined by the allowed claims of any resulting patent.

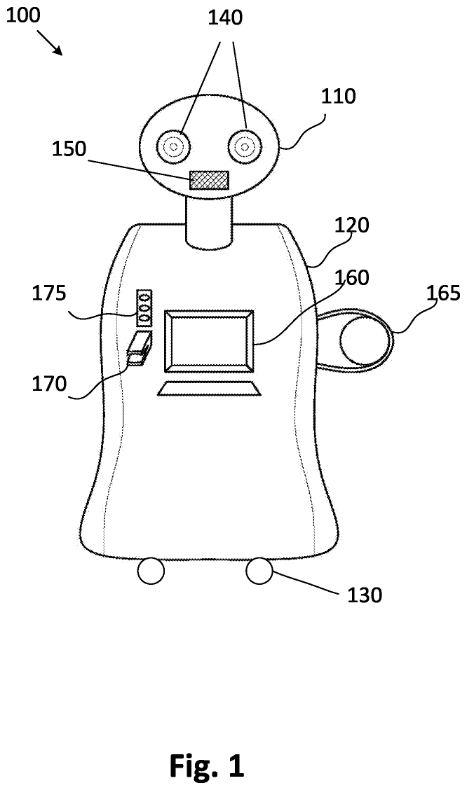

Disclosed is a robot that can be remotely controlled to take the vitals of a patient without the need of the clinical staff to come in contact with the patient. Referring to FIG. 1, which shows an exemplary embodiment of the robot 100 having a head 110, a body 120, and a wheelbase 130. The robot also includes a central processing unit that controls the operation of the robot. The central processing unit can be housed within the body or the head of the robot. The central processing unit can include at least a processor, memory, and network circuitry. The structure and functioning of the processor and the memory in computing devices are known to a skilled person. The known processor and memory can be adapted for use in the disclosed robot.

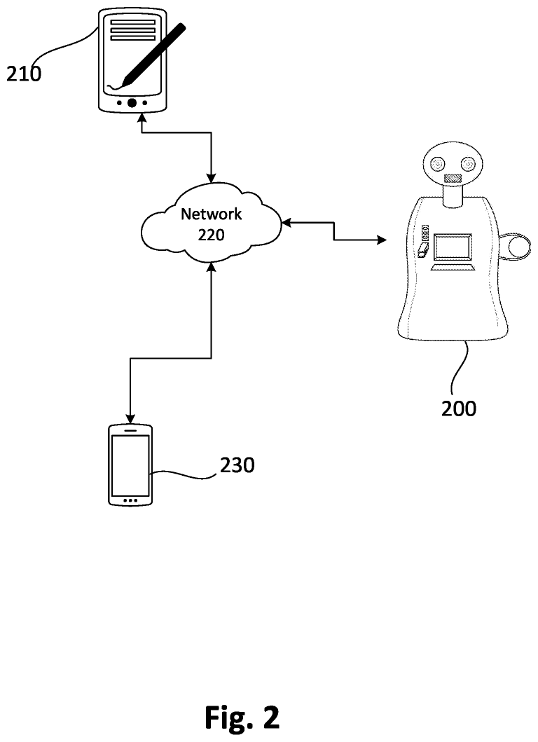

The network circuitry can permit the robot to connect to an external network for communicating with other computing devices. FIG. 2 shows the robot 200 connected to a clinical staff device 210 through the network 220. Network 220 can be wired or wireless. Suitable examples of the network include Digital Subscriber Line (DSL), coaxial cable lines, or optical fiber lines, BLUETOOTH, Wi-Fi, Worldwide Interoperability for Microwave Access (WiMAX), cellular network, 3G, 4G LTE, LTE Advanced, Mobile WiMAX, and WiMAX-Advanced. Moreover, the network can be a local area network, for example, the network of a hospital. The network can also be an isolated network connecting the robot 200 to the clinical staff device 210. Alternatively, the network can also connect to the internet. For example, FIG. 2 also shows the robot 200 connected to another device 230 through the network 220.

The clinical staff device 210 can be a desktop, workstation, laptop, tablet computer, smartphone, thin client, and like. Similarly, another device 230 can be a desktop, workstation, laptop, tablet computer, smartphone, thin client, and like. Another device can be a device of a family member, relative, friend, or any third person who wishes to communicate with the patient through the disclosed robot.

Referring to FIG. 1, the head of the robot can have a human face, and preferably the face represents a clinical professional, such as a nurse. The robot can have a head wrap that can resemble the head wrap of a clinical professional. Additionally, a stethoscope can also be provided around the neck of the robot, such that the robot itself can resemble a clinical staff to some extent. FIG. 1 shows the head 110 of the robot 100 having a pair of cameras 140. The cameras can be positioned on the face resembling the eyes of a human. Also, it is to be understood that FIG. 1 shows two cameras, the disclosed robot can have one or more than two cameras without departing from the scope of the present invention. The cameras can be operably coupled to the central processing unit. The cameras can be the eyes of the robot through which the clinical staff can see the area within the view field of the robot. The head of the robot can be rotated to view sideways without the need of rotating the whole robot. The head of the robot can be remotely rotated through a clinical staff device or remote control.

Below the camera, it can be seen a mesh forming the mouth of the robot. Behind the mesh can be a speaker 150 which resembles human speaking from the mouth. Besides, other speakers can also be provided for desired sound output.

On the front face of the body 130 can be seen a display 160 and an input. The display can be connected to the central processing unit of the robot. The display can be an LCT, TFT, and like displays known to a skilled person for displaying images and videos. An input, such as a keyboard can also be provided below the display. Alternatively, a virtual keyboard can be provided on the display. The display can have a touch interface, that can receive input from a user. The display can be used to interact with the patient. Also, the display can be used for video conferring. For example, the cameras, speaker, and display can be connected to the central processing unit, which may allow video conferencing between the patient and a person, such as a clinical staff member, a doctor, a family member, relative, or a friend. The patient can communicate with the clinical staff as well as any other person. The display can show the person on another side of the video conferring, while the sound of the person can broadcast through the speaker. The robot can also have a microphone to transmit the voice of the patient. The central processing unit can connect with the clinical staff device or any other device for video conferencing. Alternatively, the robot can show pre-recorded videos or instructions to the patient, wherein the video is on the display and sound through the speakers.

The disclosed robot can include diagnostic devices to take the vitals of a patient. The diagnostic devices can be operably connected to the central processing unit of the robot. The robot through the diagnostic devices can measure the vitals and transmit the same to the clinical staff device. The diagnostic devices can work in sync with the instructions on the display. The diagnostic devices can include a blood pressure measuring device for taking the blood pressure of the patient. FIG. 1 shows a cuff 165 on the side of the body of the robot. The display can show instructions on how to insert the hand into the cuff. The position of the cuff is such that the patient can easily insert and hold his arm in the cuff for taking the blood pressure. One or more cameras can be focused on the cuff to ensure that the hand is properly positioned into the cuff. Alternatively, sensors can be used to check the cuff around the arm.

FIG. 1 also shows a pulse oximeter 170 configured in the body of the robot. The pulse oximeter 170 can receive the pulse rate and oxygen saturation of the patient. The pulse oximeter can also be connected to the central processing unit of the robot that can transmit the diagnostic data to the clinical staff device. Additionally, can be provided an infrared thermometer to measure the temperature of the patient. The infrared thermometer 175 can also be connected to the central processing unit of the robot that can transmit the diagnostic data to the clinical staff device.

The robot can have a wheelbase, such as Arduino controlled wheels that allow the robot to move around on the floor. The robot can be programmed to take the vitals of the patient at the desired time. The robot can itself move towards the patient and take the vitals. The measurements can be then sent by the robot to the clinical staff device, without the intervention of the clinical staff. Alternately, the robot can be controlled by remote control handled by the clinical staff for taking the vitals of the patient. For example, the clinical staff member, at the safe distance from the patient, can maneuver the robot towards the patient through the remote control.

The disclosed robot can sanitize itself for preventing the spread of infection to other patients. After getting in touch with a patient, the robot can undergo a disinfection process to remove microorganisms. For example, the robot can have inbuilt UV lamps that can irradiate the areas coming in contact with the patient. Additionally, the robot can have a layer of antimicrobial composition that can kill the microorganisms and prevent their growth. In one exemplary embodiment, the outer layer of the robot can have a network of micro-showers that apply a thin layer of disinfectant liquid on the robot's outer surface and the diagnostic devices. The network of micro-showers can be connected to a reservoir containing the disinfectant liquid.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above-described embodiment, method, and examples, but by all embodiments and methods within the scope and spirit of the invention as claimed.

What is claimed is:

1. A robot for assisting clinical staff in taking vitals of a patient, the robot comprising:
a body;
a wheelbase supporting the body, the wheelbase allows the robot to move around;
a central processing unit housed in the body and operably connected to the wheelbase; and
a plurality of diagnostic devices coupled to the body, the plurality of diagnostic devices configured to measure vitals of a patient, the plurality of diagnostic devices operably coupled to the central processing unit.

2. The robot according to claim 1, wherein the robot includes a display, one or more speakers, and a microphone operably coupled to the central processing unit.

3. The robot according to claim 2, wherein the display is coupled to a front face of the body of the robot, and two cameras and one speaker are coupled to the head.

4. The robot according to claim 1, wherein the plurality of diagnostic devices includes a blood pressure measuring unit, a pulse oximeter, and an infrared thermometer.

5. The robot according to claim 4, wherein the blood pressure measuring unit is having a cuff coupled to a side of the body, the cuff configured to receive and hold an arm of the patient.

6. The robot according to claim 4, wherein the pulse oximeter is configured to receive a finger of the patient.

7. The robot according to claim 4, wherein the central processing unit is configured to:
display instructions on the display; and
operate one of the plurality of diagnostic devices for taking a measurement, wherein the instructions relate to a procedure of taking the measurement using the one of the plurality of diagnostic devices.

8. The robot according to claim 7, wherein the central processing unit is further configured to transmit the measurement to a clinical staff device.

9. The robot according to claim 1, wherein the robot further comprises a remote control, the remote control configured to remotely operate the movement and functioning of the robot.

10. The robot according to claim 1, wherein the robot includes UV lamps of irradiating the plurality of diagnostic devices.

11. The robot according to claim 1, wherein the robot includes a network of micro-shower on its outer layer and configured to apply a thin layer of a disinfectant on the outer layer and the plurality of diagnostic devices.
